(19) United States
(12) Patent Application Publication
(10) Pub. No.: US 2026/0345990 A1
(43) Pub. Date: Nov. 5, 2026

(54) CIRCULATORY SUPPORT SYSTEMS FOR USE WITH EXTRACORPOREAL BLOOD PUMPS

(71) Applicant: TC1 LLC, St. Paul, MN (US)

(72) Inventors: John Hai Nguyen, San Ramon, CA (US); Daniel I. Harjes, Carlisle, MA (US); Eric Lee, Orinda, CA (US); Balakrishnan Sivaraman, Nashua, NH (US); Kyle Marxhausen, San Francisco, CA (US); Karen Masterson, Placerville, CA (US); Shane Sadler, Tracy, CA (US); Joseph P. Sylvester, Jr., Andover, MA (US); Mike Morado, Escalon, CA (US); Fabian Frigon Franco, Livermore, CA (US); Li Jin, Daly City, CA (US); Melissa Anenden, San Mateo, CA (US); Nicholas Donnay, Plymouth, MN (US); Michael Cordisco, Brookfield, CT (US)

(21) Appl. No.: 19/473,786

(22) PCT Filed: Apr. 12, 2024
(86) PCT No.: PCT/US2024/024396

§ 371 Date: Oct. 8, 2025

Related U.S. Application Data
(60) Provisional application No. 63/459,030, filed on Apr. 13, 2023.

(51) Int. Cl. A61M 60/109; A61M 1/36; A61M 60/38

(52) U.S. Cl. CPC A61M 60/109; A61M 1/3659; A61M 60/38

(57) ABSTRACT

A circulatory support system is provided. The circulatory support system includes an extracorporeal blood pump including an inlet and an outlet, and a bifurcated graft including a first branch, a second branch configured to be coupled to the outlet of the extracorporeal blood pump, and a distal end configured to be positioned within a patient. The system further includes a catheter including a distal end configured to be positioned within a left ventricle of the patient, and a proximal end configured to be coupled to the inlet of the extracorporeal blood pump, wherein the catheter is configured to extend through the first branch of the bifurcated graft and across an aortic valve of the patient.

### CROSS-REFERENCE TO RELATED APPLICATIONS

This application claims priority to U.S. Provisional Patent Application No. 63/459,030 filed on Apr. 13, 2023, which is incorporated by reference herein in its entirety.

### BACKGROUND OF THE DISCLOSURE

### a. Field of the Disclosure

The present disclosure relates generally to circulatory support systems, and more specifically relates to circulatory support systems for use with extracorporeal blood pumps.

### b. Background

Many types of cardiac, cardiopulmonary, and pulmonary assist devices have been developed for applications in which a patient's heart and/or lungs are incapable of providing adequate circulation and/or gas exchange. For example, a patient suffering from acute heart failure may use an extracorporeal pump or circulatory support system that pumps blood out of and back into a patient's body. As another example, a patient suffering from cardiogenic shock after an invasive cardiothoracic procedure may use an extracorporeal pump or circulatory support system that provides perfusion assistance, blood oxygenation, and blood carbon dioxide removal. Extracorporeal circulatory support systems may also be used perioperatively, for example, to bypass the patient's circulatory system and provide perfusion of oxygenated blood while open-chest or other high-risk cardiothoracic surgery is performed.

At least some known circulatory support systems may be relatively invasive, and may be relatively complicated to implant within a patient. Accordingly, it would be desirable to provide circulatory support systems that are minimally invasive, that have improved flow rates, and that enable rapid left ventricular unloading (e.g., for patients in cardiogenic shock).

### SUMMARY OF THE DISCLOSURE

In one aspect, a circulatory support system is provided. The circulatory support system includes an extracorporeal blood pump including an inlet and an outlet, an inflow lumen including i) a distal end configured to be positioned within a left ventricle of a patient and ii) a proximal end configured to be coupled to the inlet of the extracorporeal blood pump, wherein the inflow lumen is configured to extend across an aortic valve of the patient, and an outflow lumen including i) a distal end configured to be positioned within the patient and ii) a proximal end configured to be coupled to the outlet of the extracorporeal blood pump.

In another aspect, a method of assembling a circulatory support system is provided. The method includes coupling a proximal end of an inflow lumen to an inlet of an extracorporeal blood pump, wherein a distal end of the inflow lumen is configured to be positioned within a left ventricle of a patient, and wherein the inflow lumen is configured to extend across an aortic valve of the patient, and coupling a proximal end of an outflow lumen to an outlet of the extracorporeal blood pump, wherein a distal end of the outflow lumen is configured to be positioned within the patient.

In yet another aspect, a circulatory support system is provided. The circulatory support system includes an extracorporeal blood pump comprising an inlet and an outlet, and a bifurcated graft including a first branch, a second branch configured to be coupled to the outlet of the extracorporeal blood pump, and a distal end configured to be positioned within a patient. The system further includes a catheter including a distal end configured to be positioned within a left ventricle of the patient, and a proximal end configured to be coupled to the inlet of the extracorporeal blood pump, wherein the catheter is configured to extend through the first branch of the bifurcated graft and across an aortic valve of the patient.

In yet another aspect, a method of assembling a circulatory support system is provided. The method includes coupling a second branch of a bifurcated graft to an outlet of an extracorporeal blood pump, wherein a distal end of the bifurcated graft is configured to be positioned within a patient, coupling a proximal end of a catheter to an inlet of the extracorporeal blood pump, wherein a distal end of the catheter is configured to be positioned within a left ventricle of the patient, and guiding the catheter through a first branch of the bifurcated graft.

In yet another aspect, a circulatory support system is provided. The circulatory support system includes an extracorporeal blood pump including an inlet and an outlet, an outer lumen including i) a distal end configured to be positioned within a left ventricle of a patient and ii) a proximal end configured to be coupled to the inlet of the extracorporeal blood pump, wherein the outer lumen is configured to extend through a port formed in a left ventricular apex of the patient, and an inner lumen including i) a distal end configured to be positioned within an aorta of the patient, and ii) a proximal end configured to be coupled to the outlet of the extracorporeal blood pump, wherein at least a portion of the inner lumen is arranged coaxially within the outer lumen, and wherein the inner lumen is configured to extend through the port and across an aortic valve of the patient.

In yet another aspect, a method of assembling a circulatory support system is provided. The method includes coupling a second branch of a bifurcated graft to an outlet of an extracorporeal blood pump, wherein a distal end of the bifurcated graft is configured to be positioned within a patient, coupling a proximal end of a catheter to an inlet of the extracorporeal blood pump, wherein a distal end of the catheter is configured to be positioned within a left ventricle of the patient, and guiding the catheter through a first branch of the bifurcated graft.

### BRIEF DESCRIPTION OF THE DRAWINGS

FIG. 1 is a simplified schematic diagram of one embodiment of a circulatory support system.

FIG. 2 is an enlarged view of components of the circulatory support system shown in FIG. 1 within a heart of a patient.

FIG. 3 is a simplified schematic diagram of another embodiment of a circulatory support system.

FIG. 4 is an enlarged view of components of the circulatory support system shown in FIG. 3 within a heart of a patient.

FIG. 5 is a simplified schematic diagram of another embodiment of a circulatory support system.

FIG. 6 is a simplified schematic diagram of another embodiment of a circulatory support system.

FIG. 7 is a simplified schematic diagram of another embodiment of a circulatory support system.

FIG. 8 is a simplified schematic diagram of another embodiment of a circulatory support system.

FIGS. 9A-9C are schematic diagrams of different embodiments of outer and inner lumens that may be used with the circulatory support system shown in FIG. 8.

FIG. 10 is a simplified schematic diagram of another embodiment of a circulatory support system.

FIG. 11 is a simplified schematic diagram of another embodiment of a circulatory support system.

FIG. 12 is a simplified schematic diagram of the dual lumen catheter of the circulatory support system shown in FIG. 11.

### DETAILED DESCRIPTION OF THE DISCLOSURE

The present disclosure is directed to circulatory support systems and methods. A circulatory support system includes an extracorporeal blood pump comprising an inlet and an outlet, and a bifurcated graft including a first branch, a second branch configured to be coupled to the outlet of the extracorporeal blood pump, and a distal end configured to be positioned within a patient. The system further includes a catheter including a distal end configured to be positioned within a left ventricle of the patient, and a proximal end configured to be coupled to the inlet of the extracorporeal blood pump, wherein the catheter is configured to extend through the first branch of the bifurcated graft and across an aortic valve of the patient.

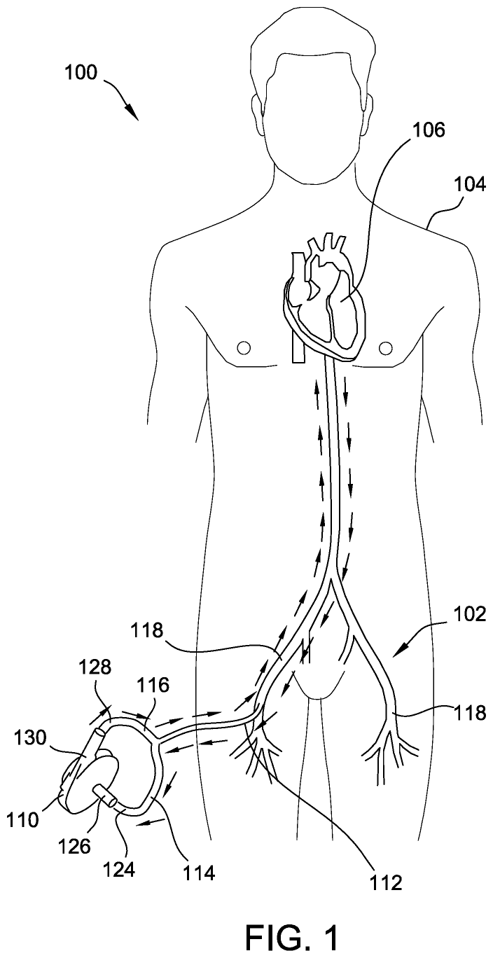

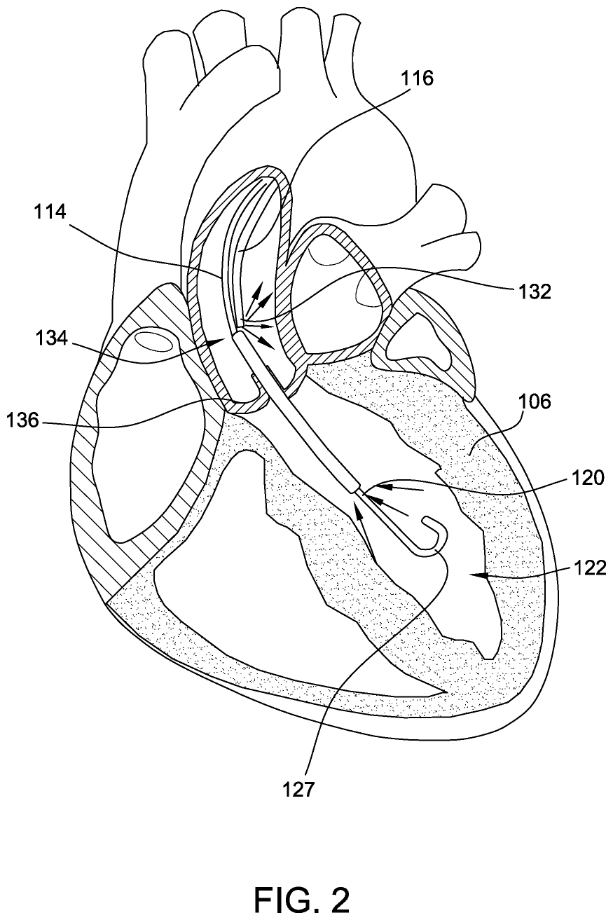

Referring now to the drawings, FIG. 1 is a simplified schematic diagram of one embodiment of a circulatory support system 100 connected to a vasculature 102 of a patient 104. FIG. 2 is an enlarged view of components of the circulatory support system 100 within a heart 106 of the patient 104.

In this embodiment, the circulatory support system 100 includes an extracorporeal blood pump 110 coupled to a dual lumen catheter 112. The dual lumen catheter 112 includes an inflow lumen 114 and an outflow lumen 116. The inflow lumen 114 and the outflow lumen 116 may be positioned side by side, or may be arranged coaxially (i.e., with one lumen located within the other lumen).

In the embodiment shown in FIG. 1, the dual lumen catheter 112 extends from the extracorporeal blood pump 110 to the heart 106 through a femoral artery 118 of the patient 104. That is, the inflow lumen 114 and the outflow lumen 116 extend through the same femoral artery 118.

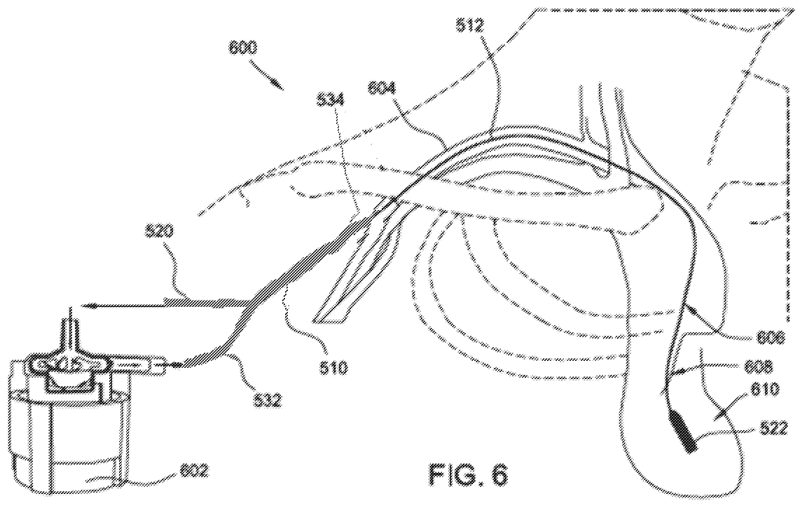

Alternatively, the inflow lumen 114 may extend through one femoral artery 118, and the outflow lumen 116 may extend through another femoral artery 118, which facilitates using larger diameter lumens (as only one lumen extends through each artery). Further, in some embodiments, at least one of the inflow lumen 114 and the outflow lumen 116 extends from the extracorporeal blood pump 110 to the heart 106 through an axillary artery (not shown) instead. For example, FIG. 6 shows a system that uses an axillary approach.

As shown in FIGS. 1 and 2, in this embodiment, a distal end 120 of the inflow lumen 114 is positioned within a left ventricle 122 of the patient 104, and a proximal end 124 of the inflow lumen 114 is coupled to an inlet 126 of the extracorporeal blood pump 110. The distal end 120 of the inflow lumen 114 may include a pigtail 127 to facilitate positioning the distal end 120 within the left ventricle 122. The pigtail 127 also prevents inflow blockage that might otherwise occur if the distal end 120 is pressed against the cardiac wall, for example. A proximal end 128 of the outflow lumen 116 is coupled to an outlet 130 of the extracorporeal blood pump 110, and a distal end 132 of the outflow lumen 116 is positioned in an aorta 134 of the patient 104. Accordingly, during operation of the circulatory support system 100, blood is pumped from the left ventricle 122 through the inflow lumen 114 into the extracorporeal blood pump 110. Subsequently, that blood is pumped from the extracorporeal blood pump 110 through the outflow lumen 116 into the aorta 134. As shown in FIG. 2, the inflow lumen 114 extends across an aortic valve 136 of the patient 104, while the outflow lumen 116 terminates in the aorta 134.

The dual lumen catheter 112 may be inserted percutaneously (i.e., through an incision) and positioned within the patient 104 using a guidewire. Alternatively, the dual lumen catheter 112 may be a steerable catheter to facilitate positioning the dual lumen catheter 112 within the patient 104.

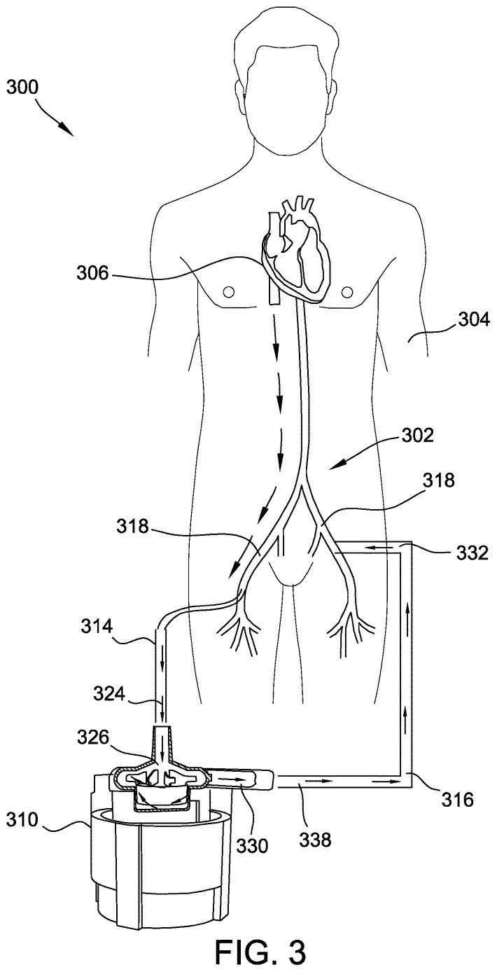

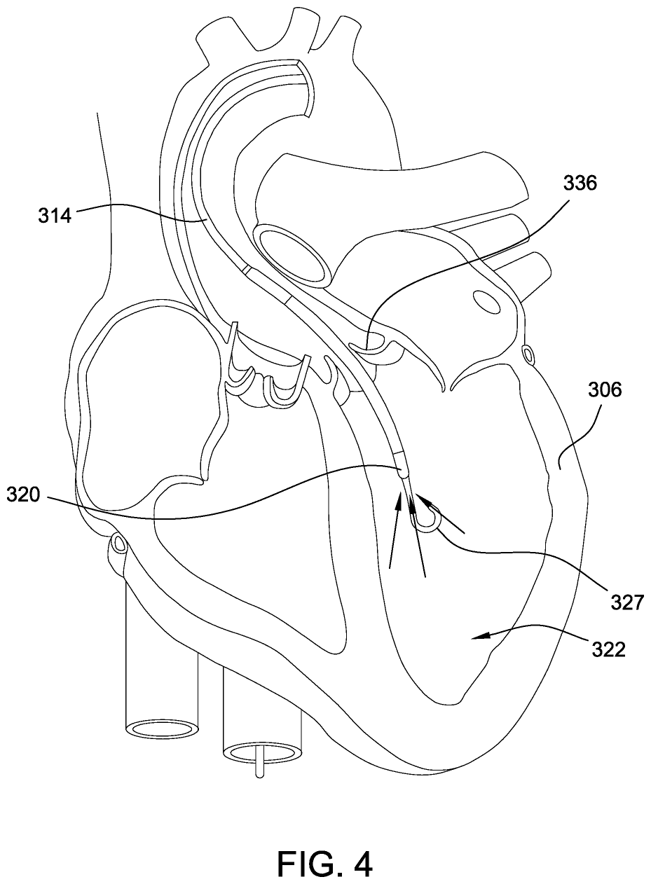

FIG. 3 is a simplified schematic diagram of another embodiment of a circulatory support system 300 connected to a vasculature 302 of a patient 304. FIG. 4 is an enlarged view of components of the circulatory support system 300 within a heart 306 of the patient 304.

In this embodiment, the circulatory support system 300 includes an extracorporeal blood pump 310 coupled to an inflow lumen 314 and an outflow lumen 316. In contrast to outflow lumen 116 of the circulatory support system 100 (shown in FIGS. 1 and 2), the outflow lumen 316 does not terminate at or near the heart 306.

In the embodiment shown in FIG. 3, the inflow lumen 314 extends from the extracorporeal blood pump 310 to the heart 106 through a femoral artery 318 of the patient 304. Alternatively, the inflow lumen 314 may extend through another artery (e.g., the axillary artery) instead (as shown, for example, in the embodiment shown in FIG. 6).

As shown in FIGS. 3 and 4, in this embodiment, a distal end 320 of the inflow lumen 314 is positioned within a left ventricle 322 of the patient 304, and a proximal end 324 of the inflow lumen 314 is coupled to an inlet 326 of the extracorporeal blood pump 310. The distal end 320 of the inflow lumen 314 may include a pigtail 327 to facilitate positioning the distal end 320 within the left ventricle 322. The pigtail 327 also prevents inflow blockage that might otherwise occur if the distal end 320 is pressed against the cardiac wall, for example. A proximal end 328 of the outflow lumen 316 is coupled to an outlet 330 of the extracorporeal blood pump 310, and a distal end 332 of the outflow lumen 316 is positioned in a femoral artery 318 of the patient 304. Accordingly, during operation of the circulatory support system 300, blood is pumped from the left ventricle 322 through the inflow lumen 314 into the extracorporeal blood pump 310. Subsequently, that blood is pumped from the extracorporeal blood pump 310 through the outflow lumen 316 into the femoral artery 318. As shown in FIG. 4, the inflow lumen 314 extends across an aortic valve 336 of the patient 304. Outputting blood from the outflow lumen 316 at a location other than an aorta of the patient 304 (in contrast to the outflow lumen 116 shown in FIG. 2) may facilitate reducing aortic regurgitation. Further, outputting blood at the femoral artery 318 facilitates using a larger diameter lumen, which may result in larger outflow, lower pressure drops, and reduced hemolysis.

In the embodiment shown, the inflow lumen 314 extends through one femoral artery 318, and the outflow lumen 316 outputs blood into another femoral artery 318. Alternatively, in some embodiments, the outflow lumen 316 may output blood into the same femoral artery 318 that includes the inflow lumen 314.

The inflow lumen 314 and the outflow lumen 316 may be inserted percutaneously (i.e., through associated incisions) and positioned within the patient 304 using guidewires. Alternatively, the inflow lumen 314 and/or the outflow lumen 316 may be implemented as a steerable catheter to facilitate positioning the inflow lumen 314 and/or the outflow lumen 316 within the patient 304.

Although FIGS. 3 and 4 depict using the circulatory support system 300 for left ventricular support, those of skill in the art will appreciate that the circulatory support system 300 may also be used for right ventricular support, bi-ventricular support, and/or left atrium support (i.e., as a left ventricle bypass). Further, the inflow lumen 314 and the outflow lumen 316 may be introduced through femoral arteries, femoral veins, jugular arteries, jugular veins, axillary arteries, and/or axillary veins as appropriate. The outflow lumen 316 may be positioned to reduce blood regurgitation near valves and/or to profuse blood advantageously along different locations on the cardiac path.

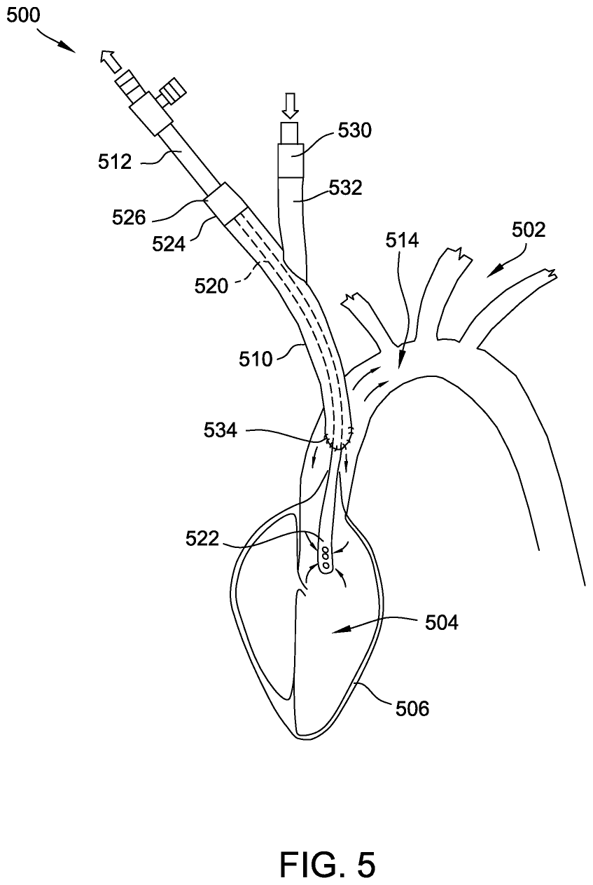

FIG. 5 is a simplified schematic diagram of another circulatory support system 500 connected to a vasculature 502 of a patient. Circulatory support system 500 is configured to unload a left ventricle 504 of the patient's heart 506 by draining blood from the left ventricle 504, passing the blood through an extracorporeal blood pump (not shown), and returning the blood to arterial portions of the vasculature 502. For example the blood may be returned to the axillary artery, the ascending aorta, or the left ventricular apex. In some embodiments an oxygenator is coupled to the extracorporeal blood pump.

As shown in FIG. 5, the circulatory support system 500 includes a bifurcated graft 510 and a catheter 512. The bifurcated graft 510 may be woven or made of, for example, expanded polytetrafluoroethylene (ePTFE). Further the bifurcated graft 510 may be sealed using animal-based or non-animal-based materials. The arrangement of the bifurcated graft 510 and the catheter 512 facilitates increasing blood flow by efficiently utilizing the large annular cross-sectional areas of the system. When used with an extracorporeal blood pump that exhibits relatively low hemolysis, the relatively large diameters of the bifurcated graft 510 and the catheter 512 enable low shear, low turbulence, and low pressure drops to achieve relatively high blood flow rates (e.g., in excess of 5 liters per minute (L/min)).

In FIG. 5, the circulatory support system 500 has a cannulation site in an ascending aorta 514, such that blood is returned to the ascending aorta 514 after passing through the extracorporeal blood pump. In this embodiment, the catheter 512 extends through a first branch 520 of the bifurcated graft 510. A distal end 522 of the catheter 512 is positioned in the left ventricle 504, and a proximal end of the catheter 512 is connected to an inlet of the extracorporeal blood pump (e.g., via suitable tubing). Accordingly, blood is pumped out of the left ventricle 504, through the catheter 512, and into the extracorporeal blood pump. A proximal end 524 of the first branch 520 includes a sealing collar 526, and the catheter 512 extends through the sealing collar 526.

To return blood to the ascending aorta 514, a proximal end 530 of a second branch 532 of the bifurcated graft 510 is connected to an outlet of the extracorporeal blood pump (e.g., via suitable tubing). Further, a distal end 534 of the bifurcated graft 510 is positioned within the ascending aorta 514. Accordingly, blood is pumped out of the extracorporeal blood pump, through the second branch 532, and out of the distal end 534 into the ascending aorta 514. Using the bifurcated graft 510 as an outflow lumen eliminates the need for a separate, second lumen.

In one embodiment, the second branch 532 is longer than the first branch 520, and a diameter of the distal end 534 of the bifurcated graft 510 (which functions as the outlet of the circulatory support system 500) is larger than a diameter of the distal end 522 of the catheter 512 (which functions as the inlet of the circulatory support system). The diameter of the bifurcated graft 510 may be, for example, in a range from 8 to 12 millimeters (mm).

The bifurcated graft 510 may be implanted via a hemi-sternotomy, with the distal end 534 of the bifurcated graft 510 anastomosed to the ascending aorta 514. The anastomosis may be performed manually, or using an automated anastomotic connector tool capable of performing anastomosis quickly and repeatedly through relatively small minimally invasive incision sites. The bifurcated first and second branches 520 and 532 may be located external to the body.

Once the bifurcated graft 510 is in position, the distal end 522 of the catheter 512 may be positioned within the left ventricle 504 using, for example a pressure-sensing guidewire. The sealing collar 526 allows for the catheter 512 to be inserted and removed from the bifurcated graft 510 while maintaining hemostasis within the bifurcated graft 510. Further, the bifurcated graft 510 may include reinforced bend-relief features to prevent kinking and/or collapse of the bifurcated graft 510.

In some embodiments, the distal end 522 of the catheter 512 and/or the distal end 534 of the bifurcated graft 510 may include a pressure transducer for monitoring the pressure of the blood pumped into the extracorporeal blood pump and/or out of the extracorporeal blood pump. The pressure may be monitored over time (e.g., periodically or continuously). In embodiments where both the distal end 522 of the catheter 512 and the distal end 534 of the bifurcated graft 510 include a pressure transducer, the monitored blood pressure may be compared with one another (e.g., to assess heart function). Further, those of skill in the art will appreciate that similar pressure transducers may be implemented within the other embodiments described herein.

In some embodiments, two separate catheters (not shown) are used instead of the bifurcated graft 510 and the catheter 512. For example, a first catheter may be used to drain blood from the left ventricle 504 and channel that blood to the extracorporeal blood pump, and a second catheter may be used to return blood flow from the extracorporeal blood pump to the ascending aorta 514. For example, the first catheter may be guided through the right axillary artery to the left ventricle 504, and the second catheter may be guided through the left axillary artery to the ascending aorta 514.

In embodiments where the cannulation site is an axillary artery, the distal end 534 of the bifurcated graft 510 may be anastomosed to the axillary artery. Then, the distal end 522 of the catheter 512 may be guided through the bifurcated graft 510, into the axillary artery, and ultimately into the left ventricle 504.

For example, FIG. 6 is a simplified schematic diagram showing a circulatory support system 600 including an extracorporeal blood pump 602 and the bifurcated graft 510. The distal end 534 of the bifurcated graft 510 is anastomosed to an axillary artery 604. Further, the distal end 522 of the catheter 512 is guided through the bifurcated graft 510, into the axillary artery 604, into the aortic arch 606, across the aortic valve 608, and ultimately into the left ventricle 610.

In operation, blood is pumped out of the left ventricle 610, through the catheter 512 (that extends through the first branch 520 of the bifurcated graft 510), and into the extracorporeal blood pump 602. To return the blood, the extracorporeal blood pump 602 pumps the blood back into the body through the second branch 532 of the bifurcated graft 510. The second branch 532 of the bifurcated graft 510 may be arranged to channel the blood into the axillary artery 604 (e.g., where the distal end 534 of the bifurcated graft 510 is anastomosed to the axillary artery 604) and/or into the aorta (e.g., through a conduit extending through the axially artery 604 into the aorta).

Those of skill in the art will appreciate that FIG. 6 is a simplified schematic diagram of bifurcated graft 510, and that the first branch 520 and the second branch 532 are cannulas. These two cannulas extend along one another (e.g., in a side by side or coaxial arrangement) over a portion of bifurcated graft 510 and are separate from one another over another portion of bifurcated graft 510 to connect to the inlet and outlet of the blood pump 602 (as shown in FIG. 6).

For an LV apex cannulation site, a combination of a bifurcated graft and a cannula may be used. Here, however, the bifurcated graft would serve as the inflow to the extracorporeal blood pump, and would need to be reinforced to prevent any collapse due to negative pressures. The catheter, then would serve as the outflow from the extracorporeal blood pump delivering blood to the ascending aorta.

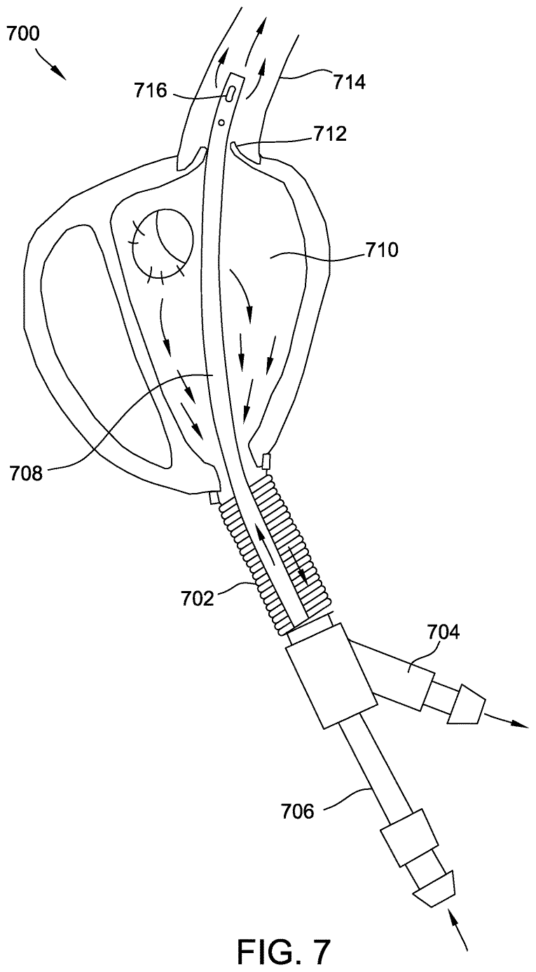

For example, FIG. 7 shows a circulatory support system 700 including a bifurcated graft 702. The bifurcated graft 702 includes a first branch 704 and a second branch 706. A catheter 708 extends through the second branch 706, through the left ventricle 710, across the aortic valve 712, and into the aorta 714. In operation, blood is pumped out of the left ventricle 710, through the first branch 704 to an extracorporeal blood pump (not shown in FIG. 7). The extracorporeal blood pump then pumps the blood through the catheter 708, and out one or more outflow openings 716 of the catheter 708 into the aorta 714.

In such embodiments, access may be gained through a small thoracotomy following an approach common for trans-apical cannulation approaches. The bifurcated graft may be anastomosed to the LV apex. Alternatively, a rigid cuff may be fixed to the LV apex, and the catheter can be passed through the cuff and secured to the cuff, The cuff itself may use sutures, staples, coils, and/or self-expanding nitinol mesh to couple to the LV apex.

After the cuff is attached, access to the left ventricle may be gained by coring the LV apex and attaching the distal end of the graft to the cuff (e.g., via an attachment ring sewn on the end of the graft). The catheter is then guided through one of the arms of the bifurcated graft, with the distal tip of the catheter positioned just distal of the aortic valve in the ascending aorta. The proximal end of the catheter would be connected to the outflow of the extracorporeal blood pump (e.g., via tubing), and the graft would be connected to the inflow of the extracorporeal blood pump (e.g., via tubing).

For longer-term support and unloading, the catheter may be removed, the bifurcated graft may be disconnected from the rigid cuff, and the bifurcated graft may be replaced with a implantable heart pump that attaches to the rigid cuff. Further, in cases where patient ambulation is desired, the circulatory support system may be paired with an extracorporeal blood pump including a relatively small motor, thereby facilitating ambulation within the hospital to support patient recovery.

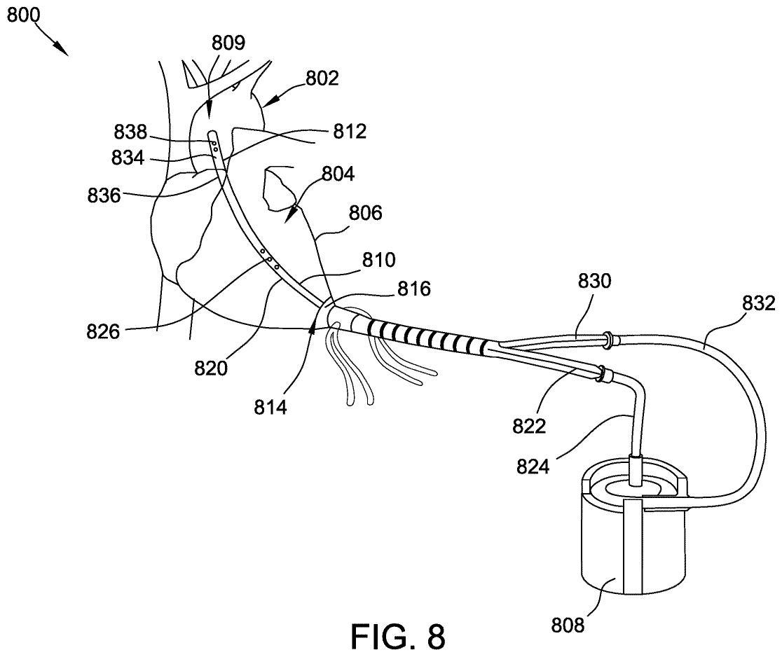

FIG. 8 is a simplified schematic diagram of another circulatory support system 800 connected to a vasculature 802 of a patient. Circulatory support system 800 is configured to unload a left ventricle 804 of the patient's heart 806 by draining blood from the left ventricle 804, passing the blood through an extracorporeal blood pump 808, and returning the blood to an ascending aorta 809 of the patient. In some embodiments an oxygenator is coupled to the extracorporeal blood pump 808.

In this embodiment, the circulatory support system 800 includes an outer lumen 810 and an inner lumen 812. Over at least a portion of the outer lumen 810, the inner lumen 812 extends within the outer lumen 810 and is concentric with the outer lumen 810, as described in more detail below. The outer lumen 810 and the inner lumen 812 are coupled to the extracorporeal blood pump 808. In some embodiments, the outer lumen 810 may be replaced with a graft.

In the embodiment shown in FIG. 8, the outer lumen 810 and the inner lumen 812 extend transapically (i.e., across a port 814 formed in an LV apex 816 of the patient). The port 814 may be formed in the patient using a left thoracotomy procedure. For example, the surgical approach and cannulation may be similar to that of transapical transcatheter valve repair and/or replacement techniques. A small left thoracotomy allows direct access to the LV apex 816, and a guidewire and modified Seldinger technique may be used to position the outer lumen 810 and the inner lumen 812.

In this embodiment, the outer lumen 810 functions as an inflow lumen. Specifically, a distal end 820 of the outer lumen 810 is positioned in the left ventricle 804, and a proximal end 822 of the outer lumen 810 is coupled to an inlet 824 of the extracorporeal blood pump 808. The distal end 820 of the outer lumen 810 defines a plurality of drainage ports 826 to facilitate draining blood from the left ventricle 804.

The inner lumen 812 functions as an outflow lumen. A proximal end 830 of the inner lumen 812 is coupled to an outlet 832 of the extracorporeal blood pump 808, and a distal end 834 of the inner lumen 812 is positioned in the ascending aorta 809. Accordingly, the inner lumen 812 extends across an aortic valve 836 of the patient. The distal end 834 of the inner lumen 812 defines a plurality of discharge ports 838 to facilitate discharging blood into the ascending aorta 809.

During operation of the circulatory support system 800, blood is drained though the draining ports 826, through the outer lumen 810, and into the extracorporeal blood pump 808. Subsequently, the blood is pumped out of the extracorporeal blood pump 808, through the inner lumen 812, and out of the discharge ports 838 into the ascending aorta 809.

The configuration of the circulatory support system 800 provides multiple advantages. For example, by using a transapical/left thoracotomy approach (as opposed to approaches through other parts of the vasculature 802), the outer and inner lumens 810 and 812 may have a relatively short length, improving manufacturability, reducing manufacturing costs, decreasing pressure drops, improving flow capacity, and reducing residence time for improved hemocompatibility. Further the relative diameters of the outer and inner lumens 810 and 812 may be adjusted to facilitate optimizing flow rates. In addition, due to relatively few bends (which results from the transapical/left thoracotomy approach) and the relatively large chamber size of the left ventricle 804, there are fewer constraints on the geometry of the drainage ports 826 and the discharge ports 838 as compared to other systems. Further, a cannula diameter can be increased behind the port 814.

Further, in cases where patient ambulation is desired, the circulatory support system may be paired with an extracorporeal blood pump including a relatively small motor, thereby facilitating ambulation within the hospital to support patient recovery.

In some embodiments, a rigid cuff (not shown in FIG. 8) maybe fixed to the LV apex 816, with the outer and inner lumens 810 and 812 passing through the cuff. The cuff may be secured using sutures, staples, coils, and/or a self-expanding nitinol mesh. The cuff offers several advantages. For example, using the cuff enables firm axial fixation of the outer and inner lumens 810 and 812, facilitates reduced bleeding, and is relatively easy to use. Further, for longer-term support and unloading, an implantable heart pump may be attached to the rigid cuff (after the outer and inner lumens 810 and 812 are removed).

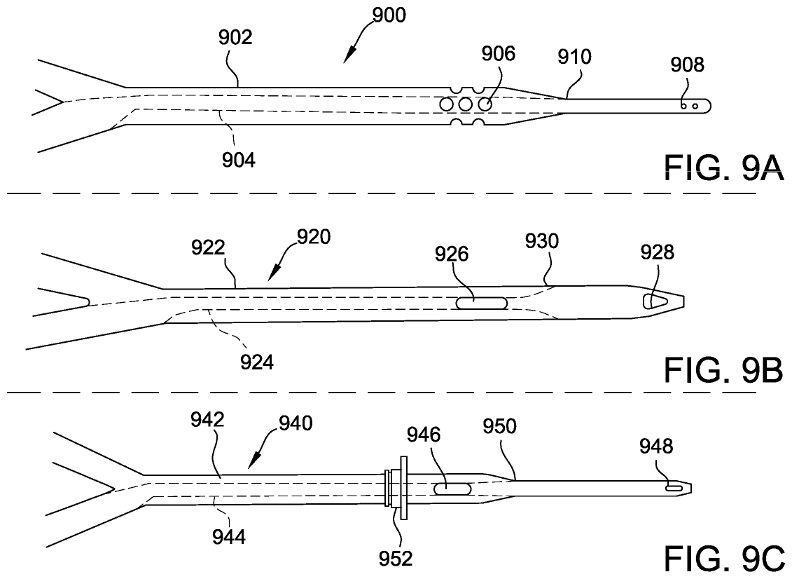

FIGS. 9A-9C are schematic diagrams of different embodiments of the outer and inner lumens. Specifically, FIG. 9A is a schematic diagram of a first lumen assembly 900. The first lumen assembly 900 includes an outer lumen 902 and an inner lumen 904. The outer lumen 902 defines a plurality of draining ports 906, and the inner lumen 904 defines a plurality of discharge ports 908.

As shown in FIG. 9A, the outer lumen 902 generally has a larger diameter than the inner lumen 904. For example, the outer lumen 902 may have a diameter of approximately 10-12 mm. However, distal of the draining ports 906, the diameter of the outer lumen 902 tapers inward to match the diameter of the inner lumen 904 at a distal end 910 of the outer lumen 902. The inner lumen 904 maintains a constant diameter and extends beyond the distal end 910 of the outer lumen 902 (i.e., across the aortic valve).

FIG. 9B is a schematic diagram of a second lumen assembly 920. The second lumen assembly 920 includes an outer lumen 922 and an inner lumen 924. The outer lumen 922 defines at least one draining port 926, and the inner lumen 924 defines at least one discharge port 928.

As shown in FIG. 9B, the outer lumen 922 generally has a larger diameter than the inner lumen 924. However, distal of the at least one draining port 926, the diameter of the inner lumen 924 tapers outward to match or exceed the diameter of the outer lumen 922 at a distal end of 930 of the outer lumen 922. The increased diameter of inner lumen 924 may reduce the rate at which blood is discharged into the ascending aorta. The inner lumen 924 extends beyond the distal end 930 of the outer lumen 932 (i.e., across the aortic valve), and tapers inward proximate the at least one discharge port 928.

FIG. 9C is a schematic diagram of a third lumen assembly 940. The third lumen assembly 940 includes an outer lumen 942 and an inner lumen 944. The outer lumen 942 defines at least one draining port 946, and the inner lumen 944 defines at least one discharge port 948.

As shown in FIG. 9C, the outer lumen 942 generally has a larger diameter than the inner lumen 944. However, distal of the at least one draining port 946, the diameter of the outer lumen 942 tapers inward to match the diameter of the inner lumen 944 at a distal end 950 of the outer lumen 942. The inner lumen 944 maintains a constant diameter and extends beyond the distal end 950 of the outer lumen 942 (i.e., across the aortic valve). Further, the outer lumen 942 is coupled to a fixation component 952, which may be configured to engage a cuff (discussed above) to fix the outer lumen 942 to the LV apex of the patient. FIGS. 9A-9C show various shapes of draining ports and discharge ports, and those of skill in the art will appreciate that any suitable shape may be used.

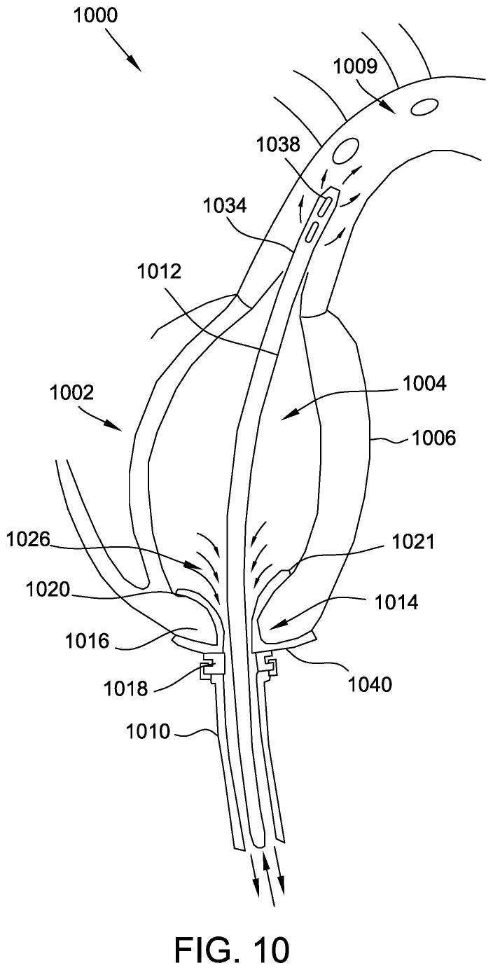

FIG. 10 is a simplified schematic diagram of another circulatory support system 1000 connected to a vasculature 1002 of a patient. Circulatory support system 1000 is similar to circulatory support system 800, except as described herein.

Circulatory support system 1000 is configured to unload a left ventricle 1004 of the patient's heart 1006 by draining blood from the left ventricle 1004, passing the blood through an extracorporeal blood pump (not shown), and returning the blood to an ascending aorta 1009 of the patient.

In this embodiment, the circulatory support system 1000 includes an outer lumen 1010 and an inner lumen 1012. Over a portion of the outer lumen 1010, the inner lumen 1012 extends within the outer lumen 1010 and is concentric with the outer lumen 1010.

As shown in FIG. 10, the outer lumen 1010 and the inner lumen 1012 extend transapically (i.e., across a port 1014 formed in an LV apex 1016 of the patient). Further, in this embodiment, a cuff 1018 is fixed to the LV apex 1016.

The outer lumen 1010 functions as an inflow lumen. Here, a distal end 1020 of the outer lumen 1010 includes an expandable mesh (e.g., Nitinol) 1021 that conforms to the inner wall of the left ventricle 1004. A proximal end of the outer lumen 1010 is coupled to an inlet of the extracorporeal blood pump. The expandable mesh 1021 defines an inlet 1026 to facilitate draining blood from the left ventricle 1004.

The inner lumen 1012 functions as an outflow lumen. A proximal end of the inner lumen 1012 is coupled to an outlet of the extracorporeal blood pump, and a distal end 1034 of the inner lumen 1012 is positioned in the ascending aorta 1009. Accordingly, the inner lumen 1012 extends across an aortic valve 1036 of the patient. The distal end 1034 of the inner lumen 1012 defines a plurality of discharge ports 1038 to facilitate discharging blood into the ascending aorta 1009.

During operation of the circulatory support system 1000, blood is drained though the inlet 1026, through the outer lumen 1010, and into the extracorporeal blood pump. Subsequently, the blood is pumped out of the extracorporeal blood pump, through the inner lumen 1012, and out of the discharge ports 1038 into the ascending aorta 1009.

In this embodiment, the outer lumen 1010 is coupled to a fixation component 1040 that engages the cuff 1018, thereby fixing a position of the outer lumen 1010 relative to the cuff 1018 (and the LV apex 1016).

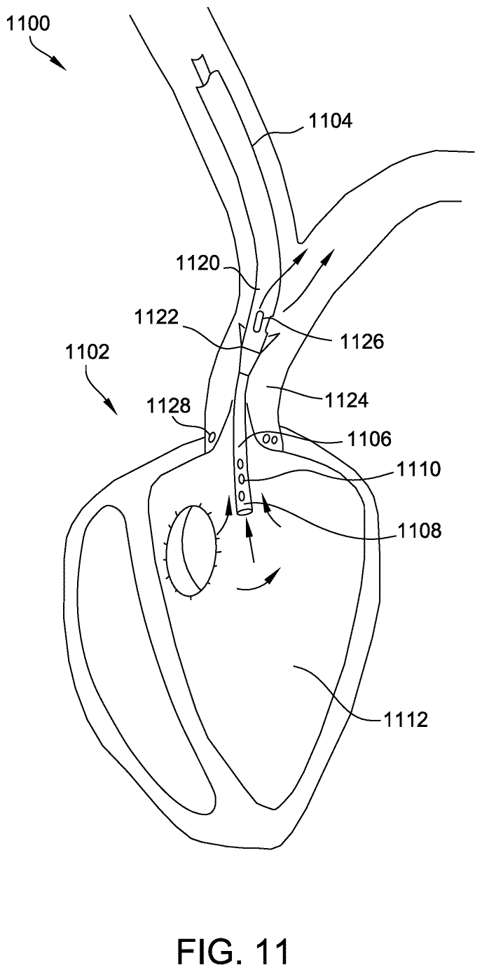

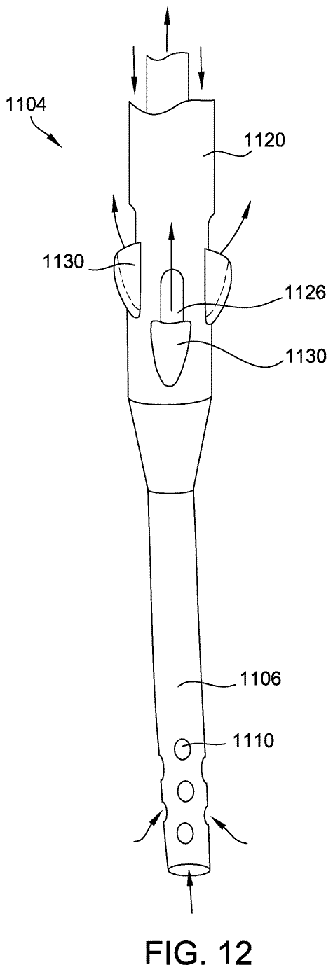

FIG. 11 is a simplified schematic diagram of another circulatory support system 1100 connected to a vasculature 1102 of a patient. Circulatory support system 1100 includes a dual lumen cannula 1104 used with an axillary approach, for example. An inner lumen 1106 of the cannula 1104 includes a distal end 1108 with one or more inlet ports 1110. The distal end 1108 is positioned in the left ventricle 1112. FIG. 12 is a simplified diagram of a portion of the dual lumen cannula 1104.

An outer lumen 1120 of the cannula 1104 includes a distal end 1122 positioned in the ascending aorta 1124. The distal end 1124 includes one or more discharge ports 1126 shaped to discharge flow backwards (i.e., in a direction away from the aortic valve 1128).

In operation, blood is pumped from the left ventricle 1112, through the inner lumen 1106 to an extracorporeal blood pump (not shown in FIG. 11). Then, the blood is returned through the outer lumen 1120 and discharged into the ascending aorta 1124 through the discharge ports 1126.

The discharge ports 1126 may have small flaps or be relatively large to facilitate discharging flow backwards. Further, as shown in FIG. 12, the outer lumen 1120 may include a bump or ridge 1130 proximate each discharge port 1126 to prevent the blood from being discharged towards the aortic valve 1128.

The systems and method described herein provide circulatory support systems and methods. A circulatory support system includes an extracorporeal blood pump comprising an inlet and an outlet, and a bifurcated graft including a first branch, a second branch configured to be coupled to the outlet of the extracorporeal blood pump, and a distal end configured to be positioned within a patient. The system further includes a catheter including a distal end configured to be positioned within a left ventricle of the patient, and a proximal end configured to be coupled to the inlet of the extracorporeal blood pump, wherein the catheter is configured to extend through the first branch of the bifurcated graft and across an aortic valve of the patient.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A circulatory support system comprising:
an extracorporeal blood pump comprising an inlet and an outlet;
an inflow lumen comprising i) a distal end configured to be positioned within a left ventricle of a patient and ii) a proximal end configured to be coupled to the inlet of the extracorporeal blood pump, wherein the inflow lumen is configured to extend across an aortic valve of the patient; and
an outflow lumen comprising i) a distal end configured to be positioned within the patient and ii) a proximal end configured to be coupled to the outlet of the extracorporeal blood pump.

2. The circulatory support system of claim 1, wherein at least a portion of the inflow lumen and a portion of the outflow lumen are positioned side by side.

3. The circulatory support system of claim 1, wherein at least a portion of the inflow lumen and a portion of the outflow lumen are arranged coaxially.

4. The circulatory support system of claim 1, wherein the inflow lumen and the outflow lumen are configured to extend through a same femoral artery of the patient.

5. The circulatory support system of claim 1, wherein the inflow lumen and the outflow lumen are configured to extend through different femoral arteries of the patient.

6. The circulatory support system of claim 1, wherein at least one of the inflow lumen and the outflow lumen is configured to extend through an axillary artery of the patient.

7. The circulatory support system of claim 1, wherein the inflow lumen and the outflow lumen are configured to be positioned within the patient percutaneously.

8. The circulatory support system of claim 1, wherein the distal end of the outflow lumen is configured to be positioned within an aorta of the patient.

9. The circulatory support system of claim 1, wherein the inflow lumen and/or the outflow lumen comprises a pressure transducer.

10. A method of assembling a circulatory support system, the method comprising:
coupling a proximal end of an inflow lumen to an inlet of an extracorporeal blood pump, wherein a distal end of the inflow lumen is configured to be positioned within a left ventricle of a patient, and wherein the inflow lumen is configured to extend across an aortic valve of the patient; and
coupling a proximal end of an outflow lumen to an outlet of the extracorporeal blood pump, wherein a distal end of the outflow lumen is configured to be positioned within the patient.

11. The method of claim 10, further comprising positioning at least a portion of the inflow lumen and a portion of the outflow lumen side by side.

12. The method of claim 10, further comprising arranging at least a portion of the inflow lumen and a portion of the outflow lumen coaxially.

13. The method of claim 10, wherein the inflow lumen and the outflow lumen are configured to extend through a same femoral artery of the patient.

14. The method of claim 10, wherein the inflow lumen and the outflow lumen are configured to extend through different femoral arteries of the patient.

15. The method of claim 10, wherein at least one of the inflow lumen and the outflow lumen is configured to extend through an axillary artery of the patient.

16. The method of claim 10, wherein the inflow lumen and the outflow lumen are configured to be positioned within the patient percutaneously.

17. The method of claim 10, wherein the distal end of the outflow lumen is configured to be positioned within an aorta of the patient.

18. The method of claim 10, wherein the distal end of the outflow lumen is configured to be positioned within a femoral artery of the patient.

19-36. (canceled)
